(19) United States
(12) Patent Application Publication
(10) Pub. No.: US 2014/0350514 A1
(43) Pub. Date: Nov. 27, 2014

(54) SYSTEMS AND METHODS FOR INTRADERMAL DELIVERY OF THERAPEUTICS USING MICRONEEDLES

(71) Applicant: NanoPass Technologies Ltd., Nes Ziona (IL)

(72) Inventor: Yotam LEVIN, Nes Ziona (IL)

(73) Assignee: NanoPass Technologies Ltd., Nes Ziona (IL)

(21) Appl. No.: 13/899,593

(22) Filed: May 22, 2013

(51) Int. Cl. A61M 37/00

(52) U.S. Cl. CPC A61M 37/0015
USPC 604/506

(57) ABSTRACT

Methods are disclosed for delivering a substance to the skin of a human subject. The methods include, obtaining a substance, such as hormones, for example, Insulin; and delivering the substance into an intradermal space within the skin of the human subject through a plurality of microneedles. The microneedles are of a height of approximately 400 micrometers to approximately 750 micrometers.

### TECHNICAL FIELD

This invention relates to administration of substances, such as insulin, into the skin.

### BACKGROUND

Insulin is currently the most effective therapy for treating diabetics. It is typically administered as a bolus and basally. Insulin is administered to patients subcutaneously (SC or SQ), through either pen injectors, hypodermic needles and syringes, or by infusion through a subcutaneously planted catheter connected to a patient-oriented pump. Bolus doses of insulin are administered, for example, at meal times, as the bolus acts rapidly, while when the patient is in a fasting state, a basal dose of insulin, administered once or twice a day, maintains the state of euglycemia in the patient.

Bolus Insulins include rapid-acting analogues with a very short Tmax (time to peak) of typically around one hour and regular insulins with a Tmax of over 2.5 hours, basal insulins have a relatively long Tmax and long half-life (e.g., Sanofi's Glargine (Lantus) is effective for about 20 hours). Combinations of basal and bolus insulins are also in use, such as those disclosed in “Long-Acting Insulin's, Insulin Detemir, Levemir®, Insulin Glargine, Lantus®,” http://www.globalrph.com/long-acting-insulins.htm.

Administration of insulin to the skin has many potential advantages including improved kinetics. Today, insulin is injected to the Subcutaneous (SC) space, using, in most cases, various devices incorporating standard metal needles and usually causing considerable pain and discomfort to the patients. Insulin injection is common, and is typically performed by the patients themselves. However, there are many limitations to this approach.

First, the inter-patient and intra-patient variability of insulin delivered into the SC space is reported to be very high, for example, as disclosed in L. Takiya, et al., “Pharmacist's Guide to Insulin Preparations: A Comprehensive Review,” Pharmacy Times CE (2005), https://secure.pharmacytimes.com/lessons/200510-03.asp. This imposes difficulties on behalf of the clinician and patient with regards to actual dosing, as well as the timing of such (before meal, during or after, with doses reflective of the anticipated calorie intake). Insulin has a narrow therapeutic range compared with other drugs, which necessitates careful dosing management, to prevent both life-risking hypoglycemia, as well as ineffective sub-dosing. Self-injection entails additional challenges, as it may be inaccurate and unreliable, and few, if any, of the available injection devices are user-friendly. Moreover, approximately 15-20% of patients are needle-phobic, making self-injection of insulin additionally challenging.

Additionally, rapid acting analogues such as Novo Nordisk's Novolog® insulin aspart, Eli Lilly's Humalog® insulin lispro, and Sanofi Aventis' Apidra® insulin glulisine, still suffer from a relatively long Tmax (time to peak), which is thought to be much longer than endogenic insulin of a healthy individual. This translates to uncomfortable and hard to manage lifestyles, which require careful planning of injection before meals, identifying and calculating the meal size and timing to prevent hypoglycemia induced by late intake of the food.

While the pharmaceutical industry has worked hard to develop “ultra rapid” analogues mimicking normal insulin secretion (with a half-life of 15 min to 35 min), the development/formulation challenge for researchers is enormous, and has not been successfully met to date. There are various approaches to accelerate the absorption of insulin into the systemic circulation including formulation, local heating, use of local hyaluronidase, etc.

However, none of the aforementioned contemporary solutions has reliably demonstrated clinical results or long term benefits. Intradermal insulin delivery, e.g., the delivery of insulin directly into the skin has shown to accelerate systemic delivery tremendously, and meet the bar of an “ultra short” analogue, with approximately 30-35 min of Tmax, and higher first hour insulin exposure, expressed as area under the curve (AUC) from time zero to one hour, expressed as AUC_{0-1 hour(h)}. However the traditional intradermal injection is difficult, unreliable and unlikely to be performed by patients themselves.

Gupta, et al, in “Rapid Pharmacokinetics of Intradermal Insulin Administered. Using Microneedles in Type 1 Diabetes Subjects,” Diabetes Technol. Ther. 2011 April: 13(4); pages 451-456 reported use of 0.9 mm length microneedles of borosilicate glass (single glass cannula) for intradermally perpendicularly injected bolus infusion of lispro insulin followed by the consumption of a standardized meal. The insulin reached peak concentrations in approximately half the time as compared to insulin administration through subcutaneous catheters.

### SUMMARY

The present invention pertains to methods and devices for controlling the pharmacokinetics of administered substances, including drug substances, such as insulin, by intradermal injection with microneedles. The invention provides a method for delivering a substance, for example, insulin, to the shallow portion of the dermis in human and other mammalian subjects. This provides enhanced pharmacokinetics and pharmacodynamics, making insulin delivery more rapid and efficient, and accordingly, more effective than the current insulin administration via conventional needles into the SC space.

However, it remains desired for insulin to be injected intradermally (ID). This is due to faster and increased absorption through the increased vascularity and lymphatics of the skin, when compared to absorption in the subcutaneous (SC) space.

Examples of substances that may be delivered in accordance with the present invention include pharmaceutically or biologically active substances including diagnostic agents, drugs, and other substances which provide therapeutic or health benefits such as, for example, nutriceuticals.

Potential diagnostic substances useful with the present invention include macromolecular substances such as, for example, inulin, ACTH (e.g., corticotropin injection), luteinizing hormone-releasing hormone (e.g., Gonadorelin Hydrochloride), growth hormone-releasing hormone (e.g. Sermorelin Acetate), cholecystokinin (Sincalide), parathyroid hormone (PTH) and fragments thereof (e.g. Teriparatide), thyroid releasing hormone and analogs thereof (e.g., protirelin), secretin, other hormones, and the like.

Therapeutic substances that may be used with the present invention include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies (mAbs); Pegylated antibodies, Pegylated proteins or any proteins modified with hydrophilic or hydrophobic polymers or additional functional groups, Fusion proteins, Single chain antibody fragments or the same with any combination of attached proteins, macromolecules, or additional functional groups thereof, Small Interfering RNA (siRNA), Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne Japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major therapeutics such as agents for the common cold, pain migraines, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

Substances may be delivered by bolus, metered bolus or infusion, through the microneedle arrays detailed below. Substances may also be delivered by infusion with pumps through the microneedles detailed below.

An embodiment of the invention is directed to a method for delivering a substance to the skin of a human subject. The method comprises: obtaining a substance selected from the group consisting of hormones; and delivering the substance into an intradermal space within the skin of the human subject through a plurality of microneedles, the microneedles of a height of approximately 400 micrometers to approximately 750 micrometers.

Another embodiment of the invention is directed to a substance selected from the group consisting of hormones, for use in the treatment or prevention of hormone deficiency. The substance is delivered into an intradermal space within the skin of the human subject through a plurality of microneedles, the microneedles of a height of approximately 400 micrometers to approximately 650 micrometers.

### BRIEF DESCRIPTION OF THE DRAWINGS

Attention is now directed to the drawings where like numerals or characters represent like or corresponding components. In the drawings:

FIG. 1A shows a series of microneedles in an array;

FIGS. 1B and 1C show side views of a delivery device with the microneedles of FIG. 1A;

FIG. 1D shows a side view of the delivery device including the fluid flow system of the delivery device;

FIG. 2A is a perspective view of a device which supports the microneedles for intradermal delivery of substances in accordance with the invention;

FIG. 2B is a side view of the device of FIG. 2A;

FIG. 3 is an exploded view of the device of FIG. 2A;

FIG. 4 is a cross-sectional view taken along line 4-4 of FIG. 2B;

FIG. 5 is a perspective view of the device of FIG. 2A showing the microneedles in detail;

FIG. 6 is a side view of another device which supports the microneedles for intradermal delivery of substances in accordance with the invention;

FIG. 7 is an exploded view of the device of FIG. 6;

FIG. 8 is a cross-sectional view taken along line 8-8 of FIG. 6;

FIG. 9 is a perspective view of the device of FIG. 6 showing the microneedles in detail;

FIGS. 10A-10C are tables of Pharmacokinetic (PK) Parameters for Diabetes Mellitus (DM) subjects and healthy subjects;

FIG. 10D is a table of p values for MicronJet® (MJ) injection of the invention compared to subcutaneous injection;

FIGS. 11A-11C are corresponding graphs for pharmacokinetics for each chart of FIGS. 10A-10C, respectively; and

FIG. 12 is a Pharmacokinetic (PK) versus Pharmacodynamic (PD) graph for fasting Diabetes Mellitus (DM) subjects.

### DETAILED DESCRIPTION

The present invention provides for delivery of insulin and other substances, such as drugs and therapeutic agents to human or animal subjects through one or more microneedles, which penetrate the skin to the depth of the intradermal space. Insulin infused as herein described through the microneedles described herein, has been found to exhibit pharmacokinetics better than that for the same substance administered by subcutaneous injection with conventional needles. Additionally, the injection by the disclosed microneedles is painless and non-intimidating for the patient and provides a consistent dose of insulin injected at a consistent delivery depth.

The injection device, also referred to as the delivery device, is used for intradermal delivery of the pharmaceutical composition, drug, agent, or the like. It provides for delivery of the pharmaceutical composition, drug, agent, or the like via one or more microneedles, intradermally (ID).

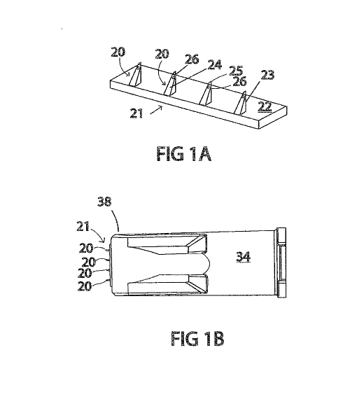

FIG. 1A shows each of the microneedles 20 in an array 21. Each microneedle 20 of the array 21 is formed, for example, of etched silicon, from a pure silicon single crystal, and has a pyramid design, and a bevel-down orientation. Each microneedle 20 includes a substrate surface 22, with a perpendicular (upright) surface 23 and an inclined (oblique) surface 24, joining at an edge 25 (the intersection of the upright 23 and oblique 24 surfaces, the oblique surface extending a majority of the height of each microneedle 20). A channel 26 or fluid flow bore, for delivering the injectable, e.g., the substance, pharmaceutical composition, drug, agent, for example, in a bolus, for injection emerges through the inclined (oblique) surface 24. The channel 26 is offset from the edge 25, so as to be non-clogging and non-leaking. (The aforementioned perpendicular (upright) 23 and inclined (oblique) 24 surfaces are defined relative to the substrate surface 22, from which each microneedle 20 projects.)

This structural arrangement and geometry of the microneedle 20, coupled with the microneedle 20 height of approximately 400 micrometers or microns (micrometers and microns are the same and are used interchangeably herein) to 1 mm (1000 micrometers), and, for example, 0.45 mm (45 micrometers) from the substrate surface 22, ensures consistency in delivering the pharmaceutical compositions, drugs, and agents to an intradermal depth of approximately 300 micrometers, close to the epidermal junction. The microneedles 20 are, for example, in accordance with those disclosed in commonly owned U.S. Pat. No. 6,533,949, entitled: Microneedle Structure and Production Method Therefor, the disclosure of which is incorporated by reference herein.

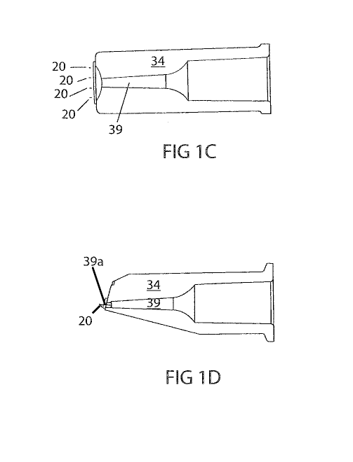

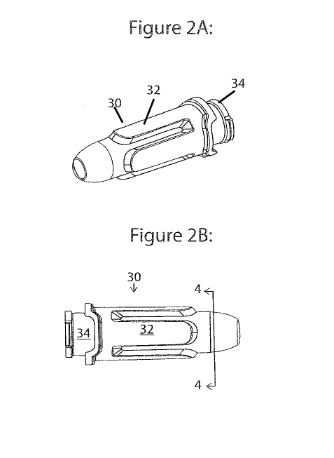

The aforementioned microneedles 20 in the array 21 are arranged on a delivery device (injector) 30, as shown in FIGS. 1B, 1C, 1D, 2A, 2B, and in the exploded view of FIG. 3. While four microneedles 20 are shown in the array 21, any number of microneedles 20 is suitable, to balance the need for a small interface touching the skin, small enough microneedles and bores, and large enough flow using the multiple channels to compensate for these. The array 21 of microneedles 20 is, for example, a linear array, which is advantageous, since penetration by the microneedles 20 is significantly more efficient and reliable, when compared to non-linear arrays (e.g., XY matrix), as detailed in commonly owned U.S. Pat. No. 8,007,466, the disclosure of which is incorporated by reference herein.

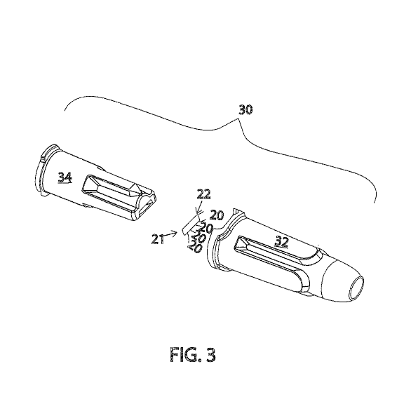

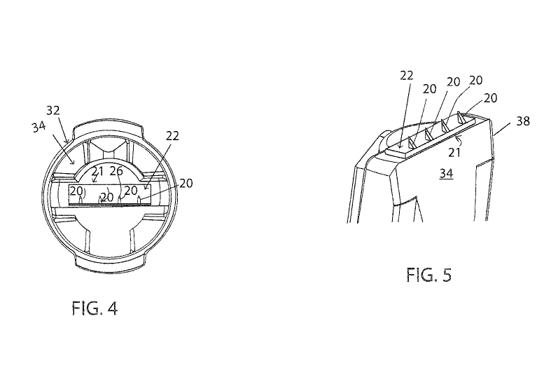

The delivery device 30 is formed of an outer member 32, which serves as a removable protective cap (removable prior to injection), which accommodates an inner member 34. The inner member 34 is slideably received and movable in the outer member 32. The inner member 34 supports the substrate surface 22 for the array 21 of microneedles 20 at its distal end 38, as shown in FIGS. 4 and 5. In FIG. 1D, wells 39 are in fluid communication with the microneedles 20 via channels 39a. These wells 39 store the injectable substance, injected by the respective microneedle 20. The microneedles 20 contact the skin by extending through the distal opening 32a of the outer member 32. The delivery device 30 is also referred to herein as the MicronJet® (MJ), and also, the MicronJet® 450, available from NanoPass Technologies LTD. of Nes Ziona 7403648 Israel.

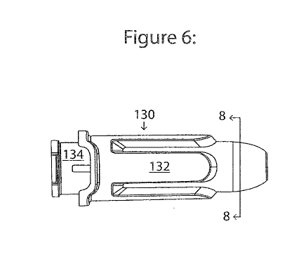

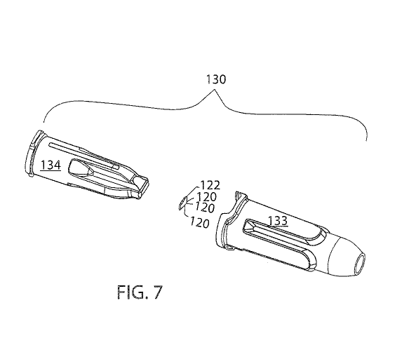

FIG. 6, and in the exploded view of FIG. 7, show an alternative delivery device 130, with microneedles 120 similar to those of the microneedles 20 detailed above, in an array 121, similar to the array 21 detailed above, except that the microneedles 120 have a height of approximately 600 microns (micrometers), similar and/or identical components to those shown in FIGS. 1A-1D, 2A, 213 and 3, above have numbers increased by “100”. While three microneedles 120 are shown in the array 121, any number of microneedles 120 is suitable.

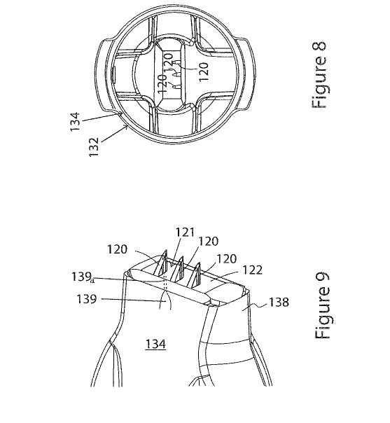

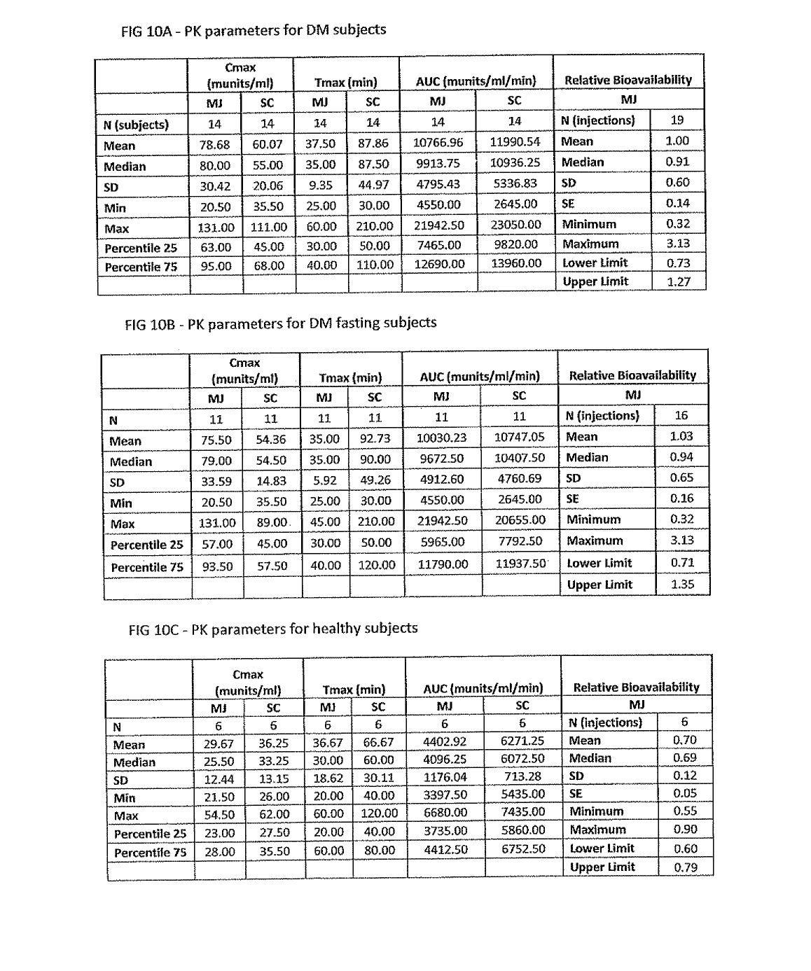

The delivery device 130, is similar to the delivery device 30 above, in that it is formed of an outer member 132, which serves as a protective cap, for a slideably received inner member 134. The inner member 134 supports the substrate surface 122 for the array 121 of microneedles 120 at its distal end 138, as shown in FIGS. 8 and 9. With the protective cap 132 removed, the microneedles 120 contact the skin. The delivery device 130 is also referred to herein as the MicronJet® (MJ) 600, available from NanoPass Technologies LTD. of Nes Ziona 7403648 Israel.

Alternative delivery devices include those in accordance with the delivery devices disclosed in commonly owned U.S. Patent Application Publication No. US 2009/0247953 A1 (U.S. patent application Ser. No. 12/096,028), entitled: Microneedle Adaptor for Dosed Drug Delivery Devices, the disclosure of which is incorporated by reference herein.

The substance, pharmaceutical composition, drug, or agent disclosed for use with the microneedles 20, 120 and delivery devices 30, 130 therefor, is for example, insulin, including insulin analogs. One exemplary insulin suitable for use with the microneedles 20, 120 and delivery devices 30, 130 disclosed herein is NovoRapid® insulin (aspart), available from Novo Nordisk A/S of Denmark, a short acting insulin analog.

Examples of substances that may be delivered in accordance with the microneedles 20 and delivery devices 30, 130 of the present invention include pharmaceutically or biologically active substances including diagnostic agents, drugs, and other substances which provide therapeutic or health benefits such as, for example, nutriceuticals.

Potential diagnostic substances useful with the present invention include macromolecular substances such as, for example, inulin, ACTH (e.g., corticotropin injection), luteinizing hormone-releasing hormone (e.g., Gonadorelin Hydrochloride), growth hormone-releasing hormone (e.g. Sermorelin Acetate), cholecystokinin (Sincalide), parathyroid hormone (PTH) and fragments thereof (e.g. Teriparatide Acetate), thyroid releasing hormone and analogs thereof (e.g., protirelin), secretin, other hormones, and the like.

Therapeutic substances that may be used with the present invention include Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies (mAbs); Pegylated antibodies, Pegylated proteins or any proteins modified with hydrophilic or hydrophobic polymers or additional functional groups, Fusion proteins, Single chain antibody fragments or the same with any combination of attached proteins, macromolecules, or additional functional groups thereof, Small Interfering RNA (siRNA), Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major therapeutics such as agents for the common cold, pain migraines, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

The microneedles 20, 120 are, for example, inserted into the skin obliquely, for an intradermal delivery. For an approximately 450-600 micron microneedle 20, 120 (extending from the substrate surface 22), the “Depth of Insertion,” the deepest point of the microneedle going into the skin, is approximately 300-500 microns. The insulin is, for example, administered as a bolus, and distributed internally, typically covering a depth of less than 1000 microns, so as to be delivered intradermally (less than 1500 microns). This administration depth of insertion or depth of injection (the “Depth of Injection” defined as the deepest point in which substance enters the skin upon injection before it is distributed) or the microneedles 20 remains intradermal, regardless of skin thickness affects such as site, body mass index (BMI) race and age. See, Laurent A, Mistretta F, Bottigioli D, Dahel K, Goujon C, Nicolas J F, Hennino A, Laurent P E, “Echographic measurement of skin thickness in adults by high frequency ultrasound to assess the appropriate microneedle length for intradermal delivery of vaccines,” Vaccine, 25(34):6423-30 (Aug. 21, 2007).

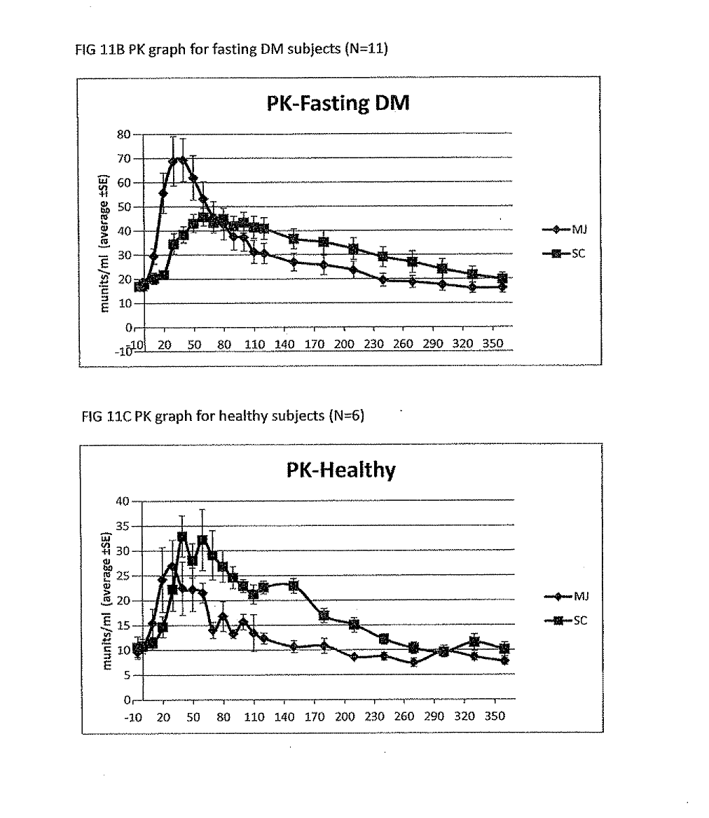

The applicants of this application conducted a study of Insulin Delivery using the microneedles 20, 120 and delivery devices 30, 130, also known as the MicronJet® delivery devices, disclosed herein with NovoRapid® insulin, in a Phase I study of healthy subjects and patients with Type II Diabetes Mellitus (DM or diabetes). The study compared insulin delivery of NovoRapid® insulin by the microneedles 20 and delivery devices 30 in intradermal administration, compared to identical doses of NovoRapid® insulin administered subcutaneously (SC) by conventional needles and syringes (collectively “needles”), for the same subject a week apart. The results for MicronJet® injection and conventional needle SC injection are provided in the graph of FIG. 11A.

While insulin is detailed herein, the devices and methods disclosed herein may be used for other pharmaceutical compositions, drugs and the like, including those as detailed herein.

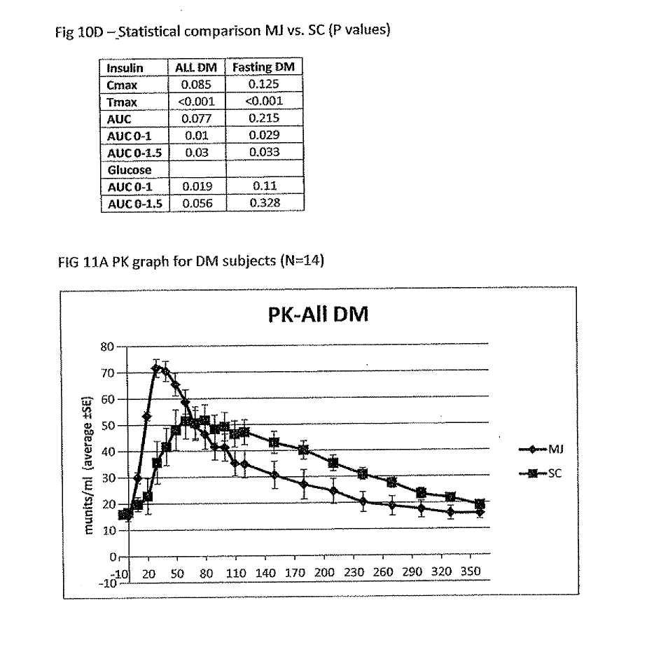

As shown in the FIG. 10A to 10D tables, the maximum insulin concentration in the blood (Cmax) tends to be higher (not statistically significant), and the Time to Peak (Tmax) in the blood is significantly shorter (p<0.001), for the insulin injected intradermally through the microneedles 20, than conventional SC delivery of the same formulation and doses in both all Diabetes Mellitus (DM) and fasting DM. The area under the curve (AUC) for both insulin administrations remains similar in the fasting DM group but tends to be slightly lower (p=0.077) in all DM subjects. Furthermore, Insulin AUC for the first 60 minutes and 90 minutes was significantly higher for MJ injected Insulin than SC administered Insulin (SC administered insulin is delivered through pen injectors, hypodermic needles and syringes, infusion through a catheter or pump, as detailed above) (in the all DM group Insulin median AUC_{0-1 h} was 3717.5 for MJ compared to 2389.75 for SC with p=0.01 and AUC_{0-1.5 h} was 4903.75 for MJ compared to 3718.75 for SC, with p=0.03; in the fasting DM group Insulin median AUC_{0-1 h} was 3612.5 for MJ compared to 2120 for SC with p=0.29 and AUC_{0-1.5 h} was 4645 for MJ compared to 3407.5 for SC, with p=0.033). Glucose median AUC for the first 60 minutes and 90 minutes was accordingly lower for MJ than for SC (in the all DM group Glucose AUC_{0-1 h} was 10497.8 for MJ compared to 10410.5 for SC with p=0.019, and AUC_{0-1.5 h} was 13831.7 for MJ compared to 13713.2 for SC with p=0.056, in the fasting DM group Glucose AUC_{0-1 h} was 8705 for MJ compared to 8986.5 for SC with p=0.011 and, e AUC_{0-1.5 h} was 11255 for MJ compared to 12267 for SC with p=0.328). AUC is defined as the area under the curve of insulin in the first hour or for the first 90 minutes following injection (and similarly, for glucose). The AUC of the second phase is significantly lower than SC insulin (Insulin AUC_{4-6 h} with p=0.002 for all DM subjects and p=0.009 for fasting DM subjects), reflecting lower late insulin exposure in the second phase (around 4-6 hours) and potentially suggesting the possibility to achieve lesser hypoglycemic events. T_{1/2Cmax} (defined as the time for the insulin to reach 50% of Cmax for the microneedle 20 administered insulin is significantly higher than that of SC insulin delivery (14 mins. Compared to 26 mins., with p=0.008 in all DM subjects, and, 13.5 mins. compared to 25 mins., with p=0.02, for fasting DM subjects), reflecting faster and higher insulin exposure in the immediate post-meal timeframe, relevant to the meal-generated glucose surge.

Further inter patient variability in Tmax is lower in MJ as compared with SC, as shown by the ratio between the dispersion and the central tendency measure (interquartile range IQR/median 28.6% compared to 68.6% in all DM subjects, and 28.6% as compared to 77.8% in fasting DM subjects. This may have a potential benefit of a more practical post prandial glucose response.

One reason for this may be that the consistent delivery depth of the microneedles 20, 120 compared with SC insulin delivery using standard needles, possibly in conjunction with the more preserved capillary network in the dermis, which is more consistent in size (and hence more consistent in delivery/diffusion across its vessel barrier) than the SC blood vessels which are typically of larger diameter range, and barrier (vessel wall) range, as well as having varying distance from the injection bolus.

Additionally, the levels of insulin of the late phase after administration are lower using intradermal delivery (ID). Accordingly, the propensity of hypoglycemia effected by high insulin levels at this phase of several hours post-meal, is potentially lower than SC insulin administration, making injection by the microneedles 20, 120 and delivery devices 30, 130 disclosed herein (the route/method/device) potentially safer than standard SC needle delivery.

Moreover, the aforementioned microneedle 20, 120 delivery of insulin results in a patients' ability to absorb insulin faster (shorter time to ½Cmax). This absorption from microneedles 20, 120 delivery (via delivery devices 30, 130) as disclosed herein, resembles normal pancreatic function to a greater degree than a SC needle injection pharmacokinetic (PK) profile. The ID insulin administration, as disclosed herein using the disclosed microneedles 20, 120 and delivery devices 30, 130, may result in long term biological benefits of lower average glucose levels, which may contribute to better blood glucose and end organ preservation.

### Example 1

### A Pilot Study to Assess the Safety, Pharmacokinetics (PK) and Pharmacodynamics (PD) of Insulin Injected Via MicronJet® (MJ) or Conventional Needles

Purpose—The purpose of this study was to evaluate the safety, pharmacokinetics and pharmacodynamics profile of Insulin injected by MicronJet® (MJ) delivery device, i.e., delivery device 30 detailed above, as compared to SC injection, in healthy volunteers and diabetes mellitus Type II subjects.

Study Design—An Open Label Study in Healthy Volunteers and Diabetes Mellitus Type II Subjects to Determine the Safety, Pharmacokinetics and Pharmacodynamics Profile of Insulin Injected by the MicronJet™ Device, specifically, delivery device 30 detailed above and shown in FIGS. 2A, 2B, 3, 4 and 5.

Objectives—The pharmacokinetics (PK) and pharmacodynamics (PD) of insulin Novorapid® (Novo Nordisk) injected via the MicronJet device intradermally, at an injection depth of approximately 300 microns intradermally, into the skin as the needles are 450-600 microns and inserted obliquely. The insulin was administered as a bolus of drug, which was distributed internally, typically covering a depth of 100-1000 microns.—This mode of injection was compared to conventional needles injected into the SC space.

Patient Population—The study population incorporated 2 primary types of subjects: 1) Healthy volunteers aged 18-40 years; and 2) Type 2 diabetic volunteers aged 30-70 years

Study Groups—Four Study Groups were evaluated:
Group 1—type 2 diabetic patients treated with two single insulin injections (one with MicronJet® and one with NovoPen® from Novo Nordisk A/S of Denmark) followed by standard meal (n=4; 3 included in the per-protocol (PP) population)
Group 2—type 2 diabetic patients treated with two single insulin injections (one with MicronJet® and one with NovoPen®) in fasting conditions (n=7; 6 included in the PP population)
Group 3—healthy volunteers treated with two single insulin injections (one with MicronJet® and one with NovoPen®) in fasting conditions (n=6; all included in the PP population)
Group 4—type 2 diabetic patients received four single insulin injections (two with MicronJet® and two with NovoPen®) in fasting conditions (n=6; 5 included in the PP population)

Procedure—Blood samples for glucose and insulin were collected at 5 minutes, baseline (0), and 10, 20, 30, 40, 50, 60, 70, 80, 90, 100, 110, 120, 150, 180, 210, 240, 270, 300, 330 and 360 minutes post-dose.

### Results

17 diabetic and 6 healthy subjects were enrolled; 20 subjects successfully completed the study and were included in the per-protocol (PP) analysis.

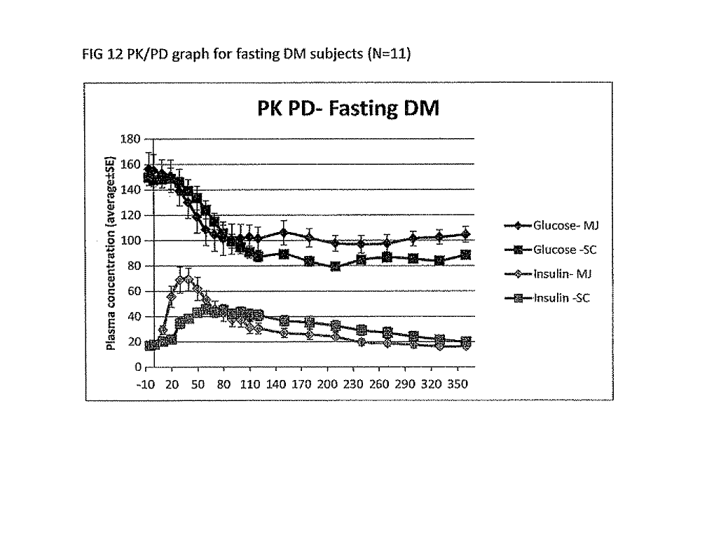

FIGS. 10A to 10C are charts of PK (Pharmacokinetic) Parameters for all diabetic subjects (N=14) in FIG. 10A, fasting diabetic subjects (N=11) in FIG. 10B, and healthy subjects (N=6) in FIG. 10C. In these figures, SD is the Standard Deviation, MJ is the MicronJet® delivery device 30, detailed above, SC is a conventional subcutaneous needle, Cmax is a maximum concentration, Tmax is time to maximum concentration, and AUC is area under the curve. FIG. 10D presents the p values of MJ compared to SC in all the parameters. Corresponding graphs for FIGS. 10A-10C, for Pharmacokinetics (PK) are represented as FIGS. 11A, 11B and 11C, and graph for Pharmacodynamics (PD), expressed as PK/PD for fasting DM group is represented as FIG. 12

The following observations were noted.

MJ has statistically significant median shorter Tmax than SC in all groups, 35 minutes as compared to 88 minutes in all diabetes patients, with p<0.001, and 35 minutes, as compared to 90 minutes in fasting diabetic patients, with p<0.001 and 30 minutes, as compared to 60 minutes in healthy subjects, with p=0.042). Shorter Tmax may have a positive effect on post-prandial glucose and may allow insulin injection post-meal and not before as currently used. MJ has statistically significant shorter time to ½Cmax than SC (14 minutes as compared to 26 minutes in all diabetes mellitus (DM), with p-0.008 subjects and 13.5 minutes as compared to 25 minutes in diabetes mellitus (DM) fasting subjects, with p=0.02). This shorter time to ½Cmax suggests an earlier glucose response.

MJ has statistically significant increased initial coverage (AUC_{0-1.5 h}) (p=0.033) and reduced late exposure (AUC_{4-6 h}) (p=0.009)—potential better glucose control and reduction in the risk of late hypoglycemic events.

The inter-patient variability in Tmax is lower in MJ when compared to SC (27% as compared to 67% in all DM, and 27% compared to 78% in fasting diabetic patients) potentially more predictable PK profile may allow better prediction of glucose effect as well as less hypoglycemic/hyperglycemic events.

Cmax was higher following MJ injection than SC in the diabetic patients (not statistically significant). This suggests a possible benefit of improved insulin coverage, with early rise and earlier decline which are more similar to endogenic insulin in healthy subjects.

Insulin AUC was similar in both injections, suggesting appropriate overall bioavailability, which is differentiated from most passive or active transdermal delivery systems for insulin; as well as from alternatives routes of administration like the inhaled, intranasal, oral delivery technologies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for delivering a substance to the skin of a human subject comprising:
obtaining a substance selected from the group consisting of hormones; and
delivering the substance into an intradermal space within the skin of the human subject through a plurality of microneedles, the microneedles of a height of approximately 400 micrometers to approximately 750 micrometers.

2. The method of claim 1, wherein the plurality of microneedles are in a linear array.

3. The method of claim 2, wherein the microneedles are of a height of approximately 450 micrometers to approximately 600 micrometers.

4. The method of claim 1, wherein the microneedles are in a bevel-down orientation.

5. The method of claim 1, wherein the microneedles include an oblique surface intersecting at least one upright surface, said oblique surface extending a majority of a height of the microneedles, and a fluid flow bore emerging through the oblique surface.

6. The method of claim 1, wherein the substance is insulin.

7. The method of claim 6, wherein the insulin includes short acting insulin analogs.

8. The method of claim 7, wherein the short acting insulin analogs include insulin aspart.

9. The method of claim 1, wherein the substance is injected as a bolus.

10. The method of claim 2, wherein the delivering the substance includes inserting the microneedles obliquely into the skin.

11. The method of claim 10, wherein the microneedles are inserted into the skin to a depth of insertion is approximately 200 micrometers to approximately 600 micrometers.

12. The method of claim 11, wherein the microneedles are inserted into the skin to a depth of insertion of approximately 300 micrometers to approximately 500 micrometers.

13.-24. (canceled)
